(19) United States
(12) Patent Application Publication
(10) Pub. No.: US 2022/0354082 A1
(43) Pub. Date: Nov. 10, 2022

(54) SOMATIC CELL METER, SOMATIC CELL MEASURING METHOD, PROGRAM, AND RECORDING MEDIUM

(71) Applicant: ADVANTEST Corporation, Tokyo (JP)

(72) Inventor: Makoto YAMAZAKI, Saitama (JP)

(73) Assignee: ADVANTEST Corporation, Tokyo (JP)

(21) Appl. No.: 17/633,371

(22) PCT Filed: Jul. 10, 2020
(86) PCT No.: PCT/JP2020/026975

§ 371 Date: Feb. 7, 2022

(30) Foreign Application Priority Data
Oct. 8, 2019 (JP) 2019-185123

(51) Int. Cl. A01J 5/013; G01N 33/04

(52) U.S. Cl. CPC A01J 5/0132; G01N 33/04

(57) ABSTRACT

According to the present invention, a somatic cell meter includes a somatic cell concentration measuring section, a time constant recording section, and a somatic cell concentration deriving section. The somatic cell concentration measuring section measures the somatic cell concentration of centrifuged raw milk in association with the duration of the centrifugation. The time constant recording section records a time constant in the association relationship between the somatic cell concentration and the duration. The somatic cell concentration deriving section derives the somatic cell concentration based on a measurement result from the somatic cell concentration measuring section and a recorded content from the time constant recording section.

### TECHNICAL FIELD

The present invention relates to measuring white blood cell in centrifuged raw milk.

### BACKGROUND ART

There has conventionally been known centrifuging raw milk. There has also been known sensors arranged to measure white blood cell in centrifuged raw milk (see Patent Literatures 1, 2, and 3, for example).

### CITATION LIST

### Patent Literature

Patent Literature 1: Japanese Patent Application Publication No. 2010-230363

Patent Literature 2: WO2010/013757

Patent Literature 3: Japanese Patent Application Publication No. 2015-508506

### SUMMARY OF THE INVENTION

### Technical Problem

However, in accordance with such related arts as described above, a prolonged period of centrifugation is required to accurately measure white blood cell in raw milk.

It is hence an object of the present invention to allow for detection of somatic cells (e.g. white blood cells or neutrophil cells) in raw milk after a shortened period of centrifugation.

### Means for Solving the Problem

According to the present invention, a somatic cell meter includes a somatic cell concentration measuring section arranged to measure the somatic cell concentration of centrifuged raw milk in association with the duration of the centrifugation; a time constant recording section arranged to record a time constant in the association relationship between the somatic cell concentration and the duration; and a somatic cell concentration deriving section arranged to derive the somatic cell concentration based on a measurement result from the somatic cell concentration measuring section and a recorded content from the time constant recording section.

According to the thus constructed somatic cell meter, a somatic cell concentration measuring section measures the somatic cell concentration of centrifuged raw milk in association with the duration of the centrifugation. A time constant recording section records a time constant in the association relationship between the somatic cell concentration and the duration. A somatic cell concentration deriving section derives the somatic cell concentration based on a measurement result from the somatic cell concentration measuring section and a recorded content from the time constant recording section.

According to the present invention, the somatic cell concentration measuring section may be arranged to measure the somatic cell concentration for a plurality of durations.

According to the present invention, the somatic cell concentration deriving section may be arranged to derive the somatic cell concentration such that an error between the measurement result from the somatic cell concentration measuring section and the association relationship is minimized.

According to the present invention, the somatic cell concentration deriving section may be arranged to derive the somatic cell concentration using a least-squares method.

According to the present invention, a somatic cell measuring method includes measuring the somatic cell concentration of centrifuged raw milk in association with the duration of the centrifugation; recording a time constant in the association relationship between the somatic cell concentration and the duration; and deriving the somatic cell concentration based on a measurement result from the measuring of the somatic cell concentration and a recorded content from the recording of the time constant.

The present invention is a program of instructions for execution by a computer to perform a somatic cell measuring process, the somatic cell measuring process including: measuring the somatic cell concentration of centrifuged raw milk in association with the duration of the centrifugation; recording a time constant in the association relationship between the somatic cell concentration and the duration; and deriving the somatic cell concentration based on a measurement result from the measuring of the somatic cell concentration and a recorded content from the recording of the time constant.

The present invention is a non-transitory computer-readable medium including a program of instructions for execution by a computer to perform a somatic cell measuring process, the somatic cell measuring process including: measuring the somatic cell concentration of centrifuged raw milk in association with the duration of the centrifugation; recording a time constant in the association relationship between the somatic cell concentration and the duration; and deriving the somatic cell concentration based on a measurement result from the measuring of the somatic cell concentration and a recorded content from the recording of the time constant.

### BRIEF DESCRIPTION OF DRAWINGS

FIG. 1 is a functional block diagram showing a configuration of a somatic cell meter 1 according to a first embodiment of the present invention;

FIG. 2 shows an association relationship between the somatic cell concentration C(t) and the duration “t”;

FIG. 3 shows a somatic cell concentration C(T) when the duration “t” of the centrifugation is equal to the time constant T; and

FIG. 4 shows somatic cell concentration measurement values C(t1), C(t2), C(t3) for durations t1, t2, t3 of the centrifugation.

### MODES FOR CARRYING OUT THE INVENTION

A description will now be given of embodiments of the present invention referring to drawings.

### First Embodiment

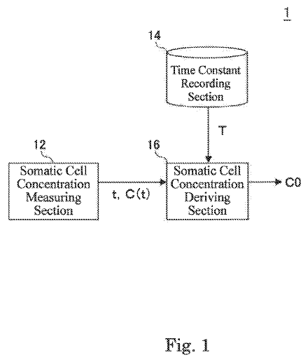

FIG. 1 is a functional block diagram showing a configuration of a somatic cell meter 1 according to a first embodiment of the present invention. The somatic cell meter 1 according to the first embodiment includes a somatic cell concentration measuring section 12, a time constant recording section 14, and a somatic cell concentration deriving section 16.

The somatic cell concentration measuring section 12 is arranged to measure the concentration C of somatic cells (e.g. white blood cells or neutrophil cells) in centrifuged raw milk in association with the duration “t” of the centrifugation. The somatic cell concentration C is a function of the duration “t” and will hereinafter be referred to as C(t).

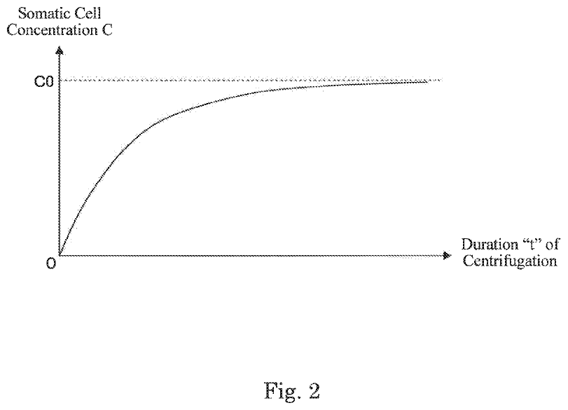

FIG. 2 shows an association relationship between the somatic cell concentration C(t) and the duration “t”. The longer the duration “t” of centrifugation of raw milk, the larger the quantity of somatic cells separated from the raw milk and thereby the higher the somatic cell concentration C(t). In due time, when all somatic cells are separated from the raw milk, the somatic cell concentration C(t) reaches a constant value C0. The constant value C0 is the somatic cell concentration of the raw milk to obtain. The somatic cell concentration C(t) is expressed by the following formula (1).

C(t)=C0(1−e^{-t/T}) (1)

The time constant recording section 14 is arranged to record a time constant T (i.e. T in the foregoing formula (1)) in the association relationship between the somatic cell concentration C(t) and the duration “t” of the centrifugation.

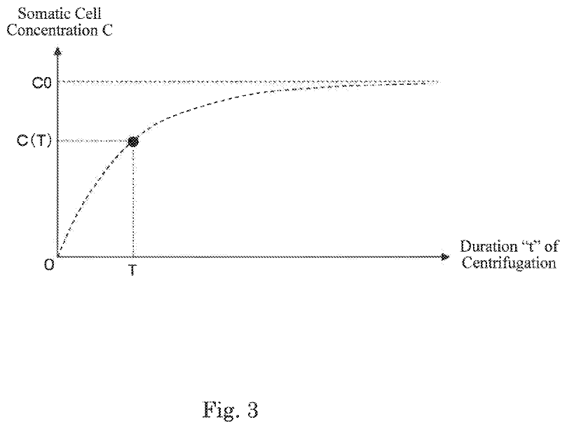

FIG. 3 shows a somatic cell concentration C(T) when the duration “t” of the centrifugation is equal to the time constant T. When the duration “t” of the centrifugation is equal to the time constant T, the somatic cell concentration C(T) has not yet reach C.

The somatic cell concentration deriving section 16 is arranged to derive the somatic cell concentration C based on a measurement result from the somatic cell concentration measuring section 12 and a recorded content (time constant T) from the time constant recording section 14. The somatic cell concentration deriving section 16 is arranged to receive a measurement result C(t) from the somatic cell concentration measuring section 12 and extract and use a value C(T) when the duration “t” of the centrifugation is equal to the time constant T for derivation of the somatic cell concentration C.

The somatic cell concentration deriving section 16 is arranged to derive the somatic cell concentration C by substituting the measurement result C(t) from the somatic cell concentration measuring section 12 into the following formula (2). The formula (2) is obtained by solving the formula (1) with T substituted into “t” for C0.

C0=C(T)/(1−e⁻¹) (2)

Next will be described an operation according to the first embodiment.

The somatic cell concentration measuring section 12 first measures the somatic cell concentration C(t) of centrifuged raw milk. According to the foregoing formula (2), the somatic cell concentration deriving section 16 derives the somatic cell concentration C based on a measurement result C(t) from the somatic cell concentration measuring section 12 and a recorded content (time constant T) from the time constant recording section 14.

In accordance with the first embodiment, it is possible to detect somatic cells (e.g. white blood cells or neutrophil cells) in raw milk after a shortened period of centrifugation during which the somatic cell concentration measurement value may not reach the somatic cell concentration C.

### Second Embodiment

In a somatic cell meter 1 according to a second embodiment, unlike the first embodiment, somatic cell concentrations C(t1), C(t2), C(t3) for multiple durations t1, t2, t3 are measured and a least-squares method is used to derive the somatic cell concentration C.

The configuration of the somatic cell meter 1 according to the second embodiment is identical to that in the first embodiment and will not be described (see FIG. 1).

The somatic cell concentration measuring section 12 is arranged to measure somatic cell concentrations C(t1), C(t2), C(t3) for multiple durations t1, t2, t3. Note, here, that the durations t1, t2, t3 are preferably equal to or shorter than the time constant T. It is noted that the somatic cell concentration measuring section 12 may be arranged to measure somatic cell concentrations for two types of durations or four or more types of durations, though arranged herein to measure somatic cell concentrations for three types of durations.

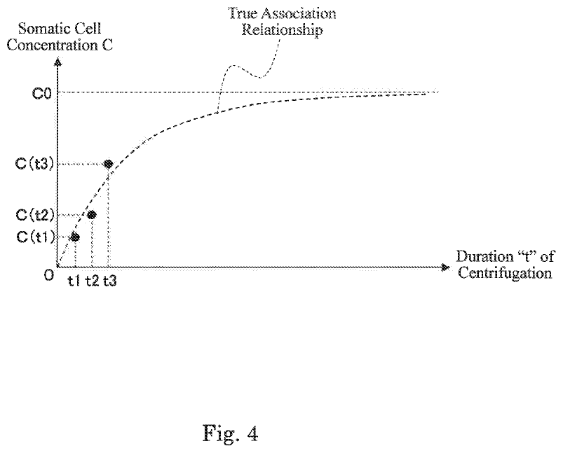

FIG. 4 shows somatic cell concentration measurement values C(t1), C(t2), C(t3) for durations t1, t2, t3 of the centrifugation. Note, here, that the association relationship between the true somatic cell concentration C(t) and the duration “t” of the centrifugation is indicated by the dotted curve. As can be seen, the measurement values C(t1), C(t2), C(t3) for the durations t1, t2, t3 all deviate from the true association relationship and have their respective errors. Also, the measurement values C(t1), C(t2), C(t3) have not yet reach C.

The time constant recording section 14 is arranged to record a time constant T.

The somatic cell concentration deriving section 16 is arranged to derive the somatic cell concentration C0 such that errors δ between the measurement results (C(t1), C(t2), C(t3)) from the somatic cell concentration measuring section 12 and the association relationship (between the somatic cell concentration C(t) and the duration “t” of the centrifugation) are minimized. Specifically, the somatic cell concentration deriving section 16 is arranged to derive the somatic cell concentration C0 using a least-squares method as follows.

The somatic cell concentration deriving section 16 is first arranged to substitute every C0 (e.g. 50,000, 100,000, 150,000, 200,000, . . . cells/milliliter) into the following formula (3) to obtain errors δn (where n=1, 2, 3) for the durations t1, t2, t3.

δn=C(tn)−C0(1−e^{-tn/T}) (3)

The somatic cell concentration deriving section 16 is further arranged to find the square root of the sum of the squares of the errors δn for the durations t1, t2, t3 as an error δ. Specifically, the errors δn are substituted into the following formula (4) to obtain the error δ.

δ=(Σₙ₌₁³δn²)^{1/2} (4)

The somatic cell concentration deriving section 16 is last arranged to obtain the somatic cell concentration C0 at which the error δ is minimized.

Next will be described an operation according to the second embodiment.

The somatic cell concentration measuring section 12 first measures the somatic cell concentrations C(t1), C(t2), C(t3) of centrifuged raw milk. According to the foregoing formula (3), the somatic cell concentration deriving section 16 obtains errors δn (where n=1, 2, 3) for the durations t1, t2, t3 at every C0 based on measurement results C(t1), C(t2), C(t3) from the somatic cell concentration measuring section 12 and a recorded content (time constant T) from the time constant recording section 14. The somatic cell concentration deriving section 16 further finds the square root of the sum of the squares of the errors δn as an error δ (see formula (4)). The somatic cell concentration deriving section 16 obtains the somatic cell concentration C0 at which the error δ is minimized.

The second embodiment exhibits the same advantageous effects as the first embodiment. In addition, using the measurement results C(t1), C(t2), C(t3) for the multiple durations t1, t2, t3 allows the somatic cell concentration C0 to be obtained more accurately compared to the first embodiment.

Incidentally, the above-described embodiments may be achieved as follows. A computer including a CPU, a hard disk, and a medium (USB memory, CD-ROM, or the like) reading device is caused to read a medium with a program recorded thereon that achieves the above-described components (e.g. the somatic cell concentration measuring section 12, the time constant recording section 14, and the somatic cell concentration deriving section 16) and install the program in the hard disk. The above-described features can also be achieved in this manner.

### DESCRIPTION OF REFERENCE NUMERAL

1 Somatic Cell Meter
12 Somatic Cell Concentration Measuring Section
14 Time Constant Recording Section
16 Somatic Cell Concentration Deriving Section
C(t) Somatic Cell Concentration
t Duration of Centrifugation
T Time Constant

1. A somatic cell meter comprising:
a somatic cell concentration measuring section arranged to measure the somatic cell concentration of centrifuged raw milk in association with the duration of the centrifugation;
a time constant recording section arranged to record a time constant in the association relationship between the somatic cell concentration and the duration; and
a somatic cell concentration deriving section arranged to derive the somatic cell concentration based on a measurement result from the somatic cell concentration measuring section and a recorded content from the time constant recording section.

2. The somatic cell meter according to claim 1, wherein
the somatic cell concentration measuring section is arranged to measure the somatic cell concentration for a plurality of durations.

3. The somatic cell meter according to claim 2, wherein
the somatic cell concentration deriving section is arranged to derive the somatic cell concentration such that an error between the measurement result from the somatic cell concentration measuring section and the association relationship is minimized.

4. The somatic cell meter according to claim 3, wherein
the somatic cell concentration deriving section is arranged to derive the somatic cell concentration using a least-squares method.

5. A somatic cell measuring method comprising:
measuring the somatic cell concentration of centrifuged raw milk in association with the duration of the centrifugation;
recording a time constant in the association relationship between the somatic cell concentration and the duration; and
deriving the somatic cell concentration based on a measurement result from the measuring of the somatic cell concentration and a recorded content from the recording of the time constant.

6. (canceled)

7. A non-transitory computer-readable medium including a program of instructions for execution by a computer to perform a somatic cell measuring process, the somatic cell measuring process comprising:
measuring the somatic cell concentration of centrifuged raw milk in association with the duration of the centrifugation;
recording a time constant in the association relationship between the somatic cell concentration and the duration; and
deriving the somatic cell concentration based on a measurement result from the measuring of the somatic cell concentration and a recorded content from the recording of the time constant.
